(19) United States
(12) Patent Application Publication
(10) Pub. No.: US 2023/0256449 A1
(43) Pub. Date: Aug. 17, 2023

(54) MICRO-DROPLET DISH

(71) Applicants: POLITECNICO DI MILANO, Milano (IT); FONDAZIONE CENTRO SAN RAFFAELE, Milano (IT)

(72) Inventors: Elena BIANCHI, Milano (IT); Oronza Antonietta BOTRUGNO, Cernusco Sul Naviglio (MI) (IT); Gabriele Angelo DUBINI, Milano (IT); Monica PIERGIOVANNI, Milano (IT); Giovanni TONON, Cernusco sul Naviglio (MI) (IT); Paola DE STEFANO, Milano (IT); Luca LEONI, Milano (IT); Pietro OLDANI, Cisliano (MI) (IT)

(73) Assignees: POLITECNICO DI MILANO, Milano (IT); FONDAZIONE CENTRO SAN RAFFAELE, Milano (IT)

(21) Appl. No.: 17/996,982

(22) PCT Filed: Apr. 28, 2021
(86) PCT No.: PCT/IB2021/053496

§ 371 Date: Oct. 24, 2022

(30) Foreign Application Priority Data
Apr. 29, 2020 (IT) 102020000009442

(51) Int. Cl. B01L 3/00

(52) U.S. Cl. CPC B01L 3/5088; B01L 3/5085; B01L 2300/161; B01L 2300/0829

(57) ABSTRACT

A micro-droplet plate including at least one fixed element which is a rigid flat support which has an upper face and a lower face, having, on said upper face, wells connected together by microchannels, said wells being delimited by a hydrophobic rim and crossed by a hole, where said wells and said microchannels define at least one microfluidic circuit; and moving elements, received in said wells through said holes, which are cylinders having an upper base and a lower base. The moving elements move, even independently of each other, along their vertical axis, and the upper base of said moving elements includes a hydrophilic surface with relief items.

### BACKGROUND

Accurate delivery and storage of large numbers of independent droplets, containing bioreagents and cellular material, is one of the crucial steps for conducting reliable large-scale biological assays.

There is a need for biological tests faster and easier to perform, to make processes more economical, while maintaining their accuracy and reproducibility. This is due to a rapid increase in the number of diagnostic and research molecular probes available and the advantages in term of information content of multiplexing assays across a wide range of instruments. Tests using cellular samples that reproduce the microenvironment - architecture, biomechanics and biochemistry - of the tissue from which these cells derive are increasingly needed, also to allow investigating new aspects of drug resistance.

These biological tests require or are reinforced by the availability of methodologies for:
i) handling of cells / particles, analytes and reagents in liquid and gel phases;
ii) controlled seeding of fluorescent / bioluminescent molecules / cells / particles;
iii) controlled immobilization of said cells / particles for the purpose of analysis;
iv) maintenance of cell viability and cell function for periods of time sufficient for the purpose of an analysis;
v) controlled exposure to one or more active ingredients in order to evaluate their effect on the cell sample.

Important progress has been made in dispensing volumes of liquids in the order of microliters with efficiency and reproducibility, progress that has made it possible to increase the number of samples and minimize the required biological sample, an important factor especially in the case of material taken from a patient.

Organoids reproduce on a small scale and in 3D the tissue from which they derive, mimicking their physiology, pathology, and response to therapy ex vivo. For this reason, methodologies that use organoids are enjoying considerable and growing interest in the international scientific community, since they allow to reproduce complex systems in the laboratory, in a reproducible way and summarizing many prerogatives of complex organs. The properties of these systems are proving invaluable not only for studying the physiology of biological systems that were not easily and / or economically examined in the laboratory until recently, but also for various pathological conditions. A further important advantage of organoids is represented by the possibility of examining the effects of active ingredients on biological systems with high throughput and rapidity, with an enormous reduction in costs and an increase in the number of active ingredients that can be analyzed. The possibility of using organoids to develop personalized treatments offers an interesting and unique alternative in the current drug development process, which often makes use of animal models that are difficult to develop, expensive and often pose ethical problems.

However, the 3D tissue formation approaches available today are difficult to integrate into large-scale systems without losing 3D tissue functionality.

Among the proposed solutions, Frey et al. 2014 (Nature Communications 5, Article number: 4250) describe a system based on “hanging drop” technology for the formation of spheroids. The system of Frey et al., functional for biological systems in the liquid phase, however, is not applicable to cultures in solid and semi-solid media and to cultures in independent drops, as required, by way of example, for studies involving the use of organoids. In addition, physical isolation of the generated drops is not achieved in the “hanging drop” system, which precludes the possibility of creating technical replicates of the individual treatments, an essential requirement in large-scale studies.

US20170298314 describes a plate for sowing drops of fluid also in the gel phase which comprises a series of elongated posts extending from the planar surface. The geometry of said elongated posts is such as to favor the formation of drops of fluid on the top thereof, which therefore act as economical dispensers of the droplets themselves.

There is a strong need for an economic, mechanically robust system for the formation and dispensing on a large scale and uniformly of gelatinous droplets of volumes between 1 and 400 microliters, or between 4 and 50 microliters.

### DESCRIPTION

The subject of the present invention is a micro-droplet plate, where with micro-droplet plate we mean here a device suitable for the formation, isolation, seeding, possibly the maintenance in culture and the controlled recovery of volumes including between 1 and 400 microliters, or between 3 and 50 microliters of fluids, preferably between 4 and 30 microliters, even more preferably between 5 and 30 microliters, where said fluids are SOL.

### BRIEF DESCRIPTION OF THE DRAWINGS

FIG. 1: perspective view (A) of the fixed element comprised in the micro-droplet plate and (B) of the micro-droplet plate according to an embodiment.

FIG. 2: perspective view (A) bottom and (B) top of a micro-droplet plate according to a further embodiment.

FIG. 3: (A) - (H) hydrophilic surfaces with relief items, different embodiments. For each embodiment, perspective view (above) and top view (below).

FIG. 4: detail of a moving element of the micro-droplet plate in three successive operating phases. (A) phase 1, loading face up (top) and, alternatively, face down (bottom) with micro-droplet formation; (B) phase 2, micro-droplet isolation; (C) phase 3, seeding, culture / treatment.

FIG. 5: perspective view of a micro-droplet plate in the seeding, culture / treatment phase, positioned above a multiwell plate.

FIG. 6: Exemplary photographs illustrating (A) micro-droplets isolated from a face-down multi-droplet plate; (B) A series of microwell images from a multi-well plate into which organoids with a multi-droplet plate were seeded.

FIG. 7: Cell viability of organoids grown in 384 multiwell plates measured at time 0 and at 96 hours from the start of the experiment (top) and after exposure for 96 hours to the indicated compounds (bottom): (A) organoids seeded in accordance with traditional methods; (B) organoids seeded with the multi-droplet plate according to the present invention.

FIG. 8: (A), (B) geometries of embodiments of the relief items.

FIG. 9: (A), (B), (C), (D), (E) caps of SOL formed at the base support of said moving elements, with different geometries of the relief items on the hydrophilic surfaces with relief.

FIG. 10: micro-droplet loading performed by a dispenser, alternative embodiments: (A) the fluid is released from a dispenser which does not touch the plate, the fluid reaches a defined well. The same dispenser, by translating, can proceed to supply fluid into additional wells; (B) the fluid is released from a dispenser almost in contact with the plate, the fluid is dispensed directly into a defined well.

### DETAILED DESCRIPTION

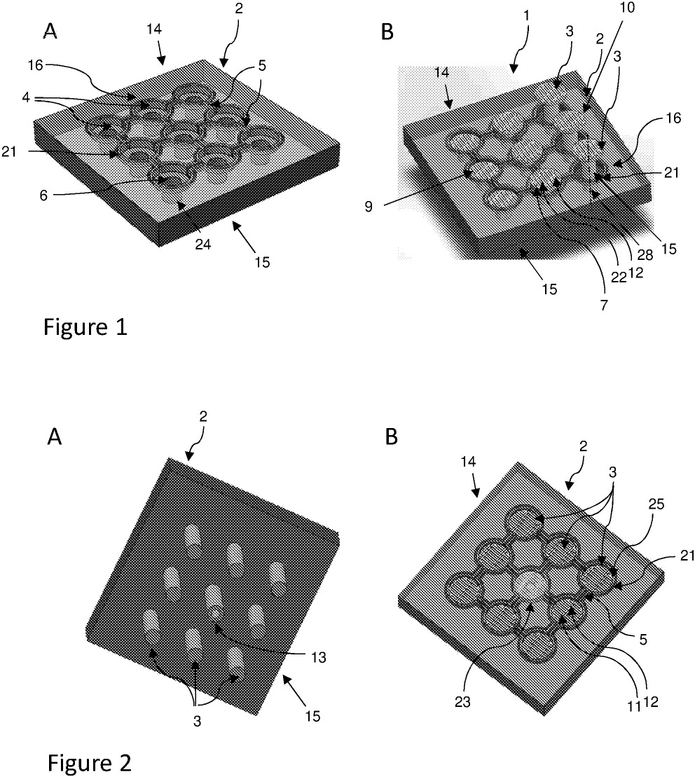

In an embodiment, shown schematically in FIG. 1, the micro-droplet plate 1 according to the present invention comprises a fixed element 2 and moving elements 3, also called pistons. Said fixed element, with reference to FIG. 1A, is a rigid flat support which has an upper face 14 and a lower face 15. On said upper face 14 there are a series of wells 4 connected to each other by microchannels 5. Each of said wells 4 is delimited by a hydrophobic rim 21. Said wells 4 are crossed by a hole 6. In one embodiment, said hole 6 occupies the entire base area of said well 4. In an alternative embodiment, said hole 6 occupies a portion of said base area of said well 4. Said base of said well 4 is hydrophilic or hydrophobic, in a preferred form it is hydrophobic. Said wells 4 and said microchannels 5 constitute a microfluidic circuit 16. In one embodiment, said wells 4 have a flat bottom; in an alternative embodiment, said wells 4 have a conical bottom, where said hole 6 occupies the vertex of said cone.

Optionally, said fixed element 2 also comprises at least one access channel 13. Said at least one access channel connects the lower face 15 of said fixed element 2 to said microfluidic circuit 16. In one embodiment, said access channel 13 is inserted in one of said microchannels 5, in an alternative form, it is inserted into one of said wells 4, for example by accessing through a side wall in one of said microchannels 5 or in one of said wells 4. In a preferred embodiment, with reference to FIGS. 2A, 2B, said access channel 13 is inserted into an access well 23, located in an almost central position of said support element.

In one embodiment, said holes 6 pass through the rigid flat support and, in correspondence with each of them, from the lower face 15 of said fixed element 2 emerges a guide channel 24. In each of said holes 6 one of said moving elements 3 is received (FIG. 1B).

Said moving elements 3 are cylinders made of plastic material which have an upper base 7 and a lower base 8. Said moving elements 3, inserted through said holes 6 in said fixed element 2, move along their vertical axis 28 and, optionally, rotate around their vertical axis 28. Once inserted in said fixed element 2, said moving elements 3, moving along their own vertical axis 28, assume, even independently of one another, variable positions between a rest position 9 and an operating position 10.

In the embodiment which comprises guide channels 24, said moving elements 3 inserted in said fixed element 2 through said holes 6, move along their own vertical axis 28 conveniently housed in said guide channels 24. Said moving elements 3, in said rest position 9, have the upper base 7 in a substantially co-planar position with the base of said well 4. That is, in said rest position 9 said upper base 7 of said moving element 3, closing said hole 6, makes said well 4 similar to a closed bottom well.

The upper base 7 of said moving element 3 comprises a hydrophilic surface with relief items 12. In an embodiment, where said moving element 3 is a cylinder with a circular base, the diameter of said upper base 7 is almost equal to the diameter of said hole 6, which will also be suitably circular. In this embodiment, it is the same upper base 7 that is a hydrophilic surface with relief items 12. In a preferred embodiment, said moving element 3 is a cylinder on whose upper base 7 of area A rests a base support 11 of area A′, where said area A′ of said base support 11 is greater than said area A of said upper base 7. In this embodiment, said moving element 3, inserted through said hole 6 in said fixed element 2, in the rest position 9, it conveniently closes said hole 6 with said base support 11. In this embodiment, said base support 11 is a hydrophilic surface with raised elements 12.

Conveniently, the closure operated by each of said moving elements 3 of each of said holes 6 is a sealing closure. Optionally, suitable gaskets are positioned on said hole 6 to favour said seal.

The micro-droplet plate 1, with said moving elements 3 in the rest position 9, therefore comprises a series of wells 4, delimited by said hydrophobic rims 21, similar to closed-bottom wells in microfluidic connection between them, the base of which consists of said hydrophilic surface with relief items 12, where said microfluidic connection is ensured by said microchannels 5.

The micro-droplet plate with said moving elements 3 in the rest position comprises an integrated microfluidic circuit 25 which includes the hydrophilic areas of said micro-droplet plate. In one embodiment, said integrated microfluidic circuit 25 consists of said microfluidic circuit 16 and said hydrophilic surfaces with relief items 12.

Said relief items 22 on said hydrophilic surface with relief items 12 are adjacent to each other and occupy a percentage between 10% and 70% of the total surface, in an embodiment between 40 and 60%, or between 15 and 50%, preferably about 20%.

Said relief items 22 are distributed in a controlled manner on said hydrophilic surfaces, i.e. according to a regular geometric arrangement, which can be homogeneous, where said relief items are the same or different from each other and arranged with the same pattern on the surface, or inhomogeneous, with relief items of different shape / size / distribution on the surface.

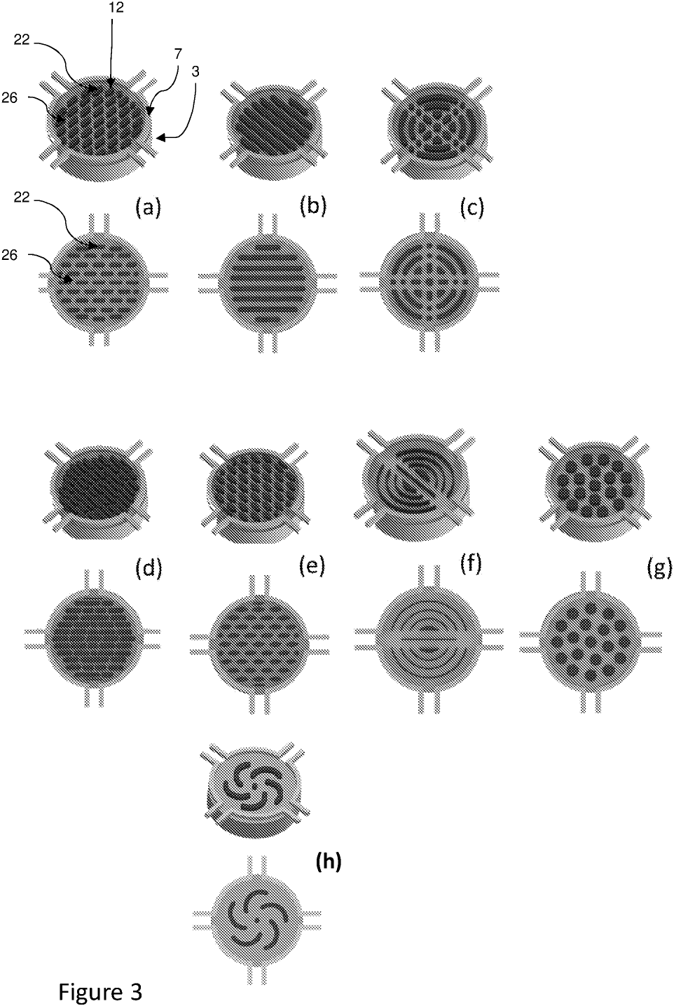

FIG. 3 shows, by way of example, some geometries of said relief items 22. In the figure, embodiments of the geometry and distribution of said relief items are schematically shown. Said relief items 22, shown in black in the figure, are surrounded by interconnected cavities 26, in grey in the figure. Advantageously, said relief items 22 do not have vertical but rounded rims. In one embodiment, said relief items 22 are elongated elements (FIGS. 3A, D, E), or circular elements (FIG. 3G) or bars (FIG. 3B), arched elements (FIG. 3F), or a set of elements with variable geometry among those indicated (FIG. 3C). In a further embodiment, they are elongated curved elements, which extend radially (FIG. 3H).

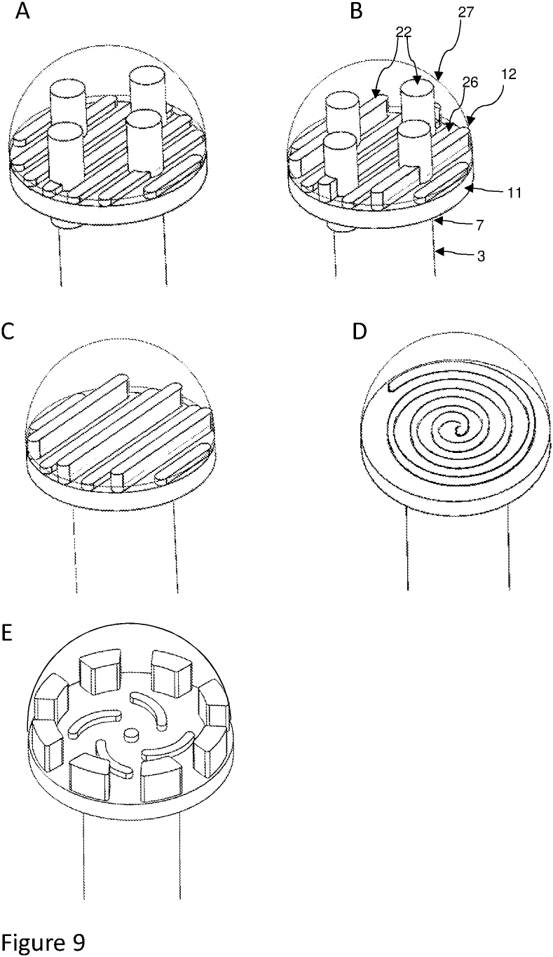

The height of said relief items 22 is approximately equal to the depth of said well 4. In one embodiment, all elements 22 on a hydrophilic surface with relief items 12 have equal height. In one embodiment, the elements 22 on a hydrophilic surface with relief items 12 have different heights independently of each other (FIGS. 9A, 9B, 9C). Preferably, the relief items 22 having a greater height are arranged on the perimeter of said hydrophilic surface (FIG. 9E). In this embodiment, said perimeter relief items 22 advantageously delimit the surface, allowing for a better control of the SOL cap 27.

Said surface from which said relief items 22 emerge is optionally subjected to a surface treatment suitable to make it hydrophilic, in the degree functional to filling the wells.

The number of said relief items 22 on said hydrophilic surfaces with relief items 12 is such as to reach the % of occupation of the surface functional for the purpose, i.e. between 10 and 70%, or between 40 and 60%, or between 15 and 50%, preferably about 20%.

In a preferred form, said relief items 22 are arched elements and elongated elements arranged on said surface along concentric circles, as shown in FIG. 3C.

In one embodiment, and with reference to FIG. 8A, geometric relief items 22 of elongated or almost circular shape have the minimum and maximum dimensions indicated in table 1, where
D = diameter or equivalent diameter
SDa = characteristic distance between one element and another in the direction
SDb = other characteristic distance between one element and another in direction, in the case of non-homogeneous distributions
LD = second characteristic dimension of the element, if it is not attributable to circular geometry
H = height of said elements.

TABLE 1

| | | | | | |
|---|---|---|---|---|---|
| | D | SDa | SDb | LD | H |
| Min | 10 µm | 10 µm | 10 µm | 10 µm | 10 µm |
| Max | 500 µm | 500 µm | 500 µm | 500 µm | 3 cm |

In one embodiment, and with reference to FIG. 8B, bar or arched geometric relief items 22 or, in accordance with FIG. 9D, spiral, have the minimum and maximum dimensions indicated in table 2, where
A is the measurement of the long side of said relief item
B is the measurement of the short side of said relief item
w is the distance of one of these elements from the neighbouring element
H is the height of said elements.

TABLE 2

| | | | | |
|---|---|---|---|---|
| | A | B | w | H |
| Min | 10 µm | 500 µm | 10 µm | 10 µm |
| Max | 500 µm | 5 cm | 500 µm | 2 mm |

A fluid which is loaded into the microfluidic circuit 16 fills the hydrophilic areas in the microfluidic circuit by capillarity, forming drops on the upper base or on the base support of said moving elements 3, where the size of said drops is defined by said hydrophobic rims 21 which delimit said wells and from the surface tension at the liquid / air interface.

The height of said relief items 22 is approximately 10-20% of the size of the drop that is formed on said hydrophilic surface with moving elements 12. By way of example, in FIGS. 9A, 9B, 9C and 9E are schematically shown said drops, which are SOL caps 27, formed in correspondence of the base support 11 which is on the upper base 7 of said mobile elements 3, where the geometric relief items 22 on said hydrophilic surface with relief items 12 have a different height from each other.

In one embodiment, said fixed element 2 and said moving elements 3 are made of a thermoplastic polymer selected from the group consisting of polystyrene, polyolefin, polycarbonate and acrylonitrile-butadiene-styrene, or it is of polysiloxane, preferably of PDMS (polydimethylsiloxane).

Said fluid with which the micro-droplet plate according to the present invention is loaded is a SOL, i.e. a fluid which, by phase transition, changes from a liquid phase to a solid one, gel.

Said phase transition occurs due to physical (temperature, UV) or chemical stimuli, such as exposure to specific ions or solvents, change in pH.

In a preferred form, said SOL is a formulation which polymerizes producing a biologically active matrix. By way of example, it is a formulation based on alginate, chitosan, hyaluronic acid, fibrin, laminin, collagen or combinations of these. Preferably, it is Matrigel (Corning® Matrigel®).

On the hydrophilic surface with raised elements 12 which is on the upper base 7 of each of said moving elements 3, when said moving element 3 is in the rest position 9, drops of said fluid SOL are obtained, named caps of SOL 27 which, after a suitable phase transition, they become gel drops 17. In one embodiment, said SOL comprises organoids 18 and they are homogeneously distributed in said gel drops 17.

Said gel drops 17, once formed on the hydrophilic surface with relief items 12 of each of said moving elements 3, are conveniently seeded in culture plates containing growth medium, moving said moving elements 3 from the rest position 9 to the operative position 10. Once placed in the culture medium, the gel drops remain, preserving within them the architecture of the organoids and at the same time ensuring the necessary exchange of metabolites between the organoids and the culture medium.

By way of example, said culture medium is added with molecules whose effect on organoids is to be tested, allowing for large-scale drug screening tests on organoids.

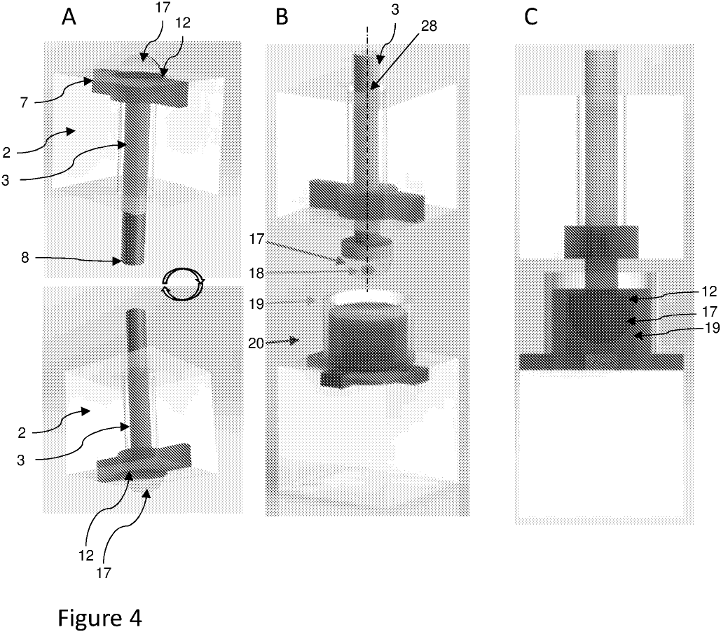

A micro-droplet plate according to the present invention is now described under conditions of use. The different operational phases are outlined in FIG. 4.

### Phase 1, Loading With Micro-Doplet Formation FIG. 4A

A micro-droplet plate according to the present invention, with said moving elements 3 in the rest position 9, is positioned face up (FIG. 4A, top panel) and the integrated microfluidic circuit 25 is loaded with a fluid, where said fluid is a SOL.

Said fluid is loaded from below, through said access channel 13, or from above. Said fluid is preferably introduced into one of the microchannels 5 or into one of the wells 4, or 23 in case of loading through said access channel 13, located in an almost central position with respect to said fixed element 2.

For top loading, by way of example, a pipette, or a syringe pump, or an injector are used, inserting them into one of the wells 4 or microchannels 5.

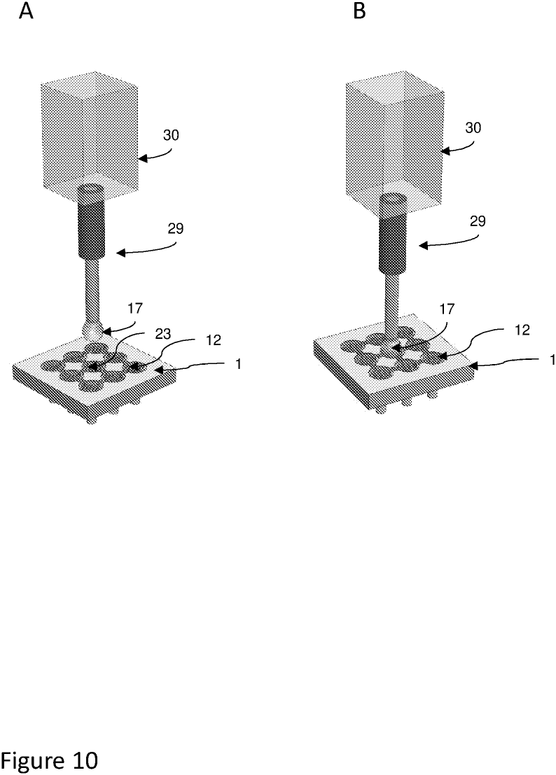

In an alternative embodiment, with reference to FIG. 10, the loading is obtained by means of a dispenser 29 in fluidic connection with a reservoir 30. Said dispenser is conveniently managed in an automated manner according to methods known to the skilled in the art, and it is positioned in such a way that the released fluid reaches the selected well (FIG. 10A). Alternatively, said dispenser is positioned near the plate, so as to release the fluid directly into the selected well (FIG. 10B). The automated movement of the dispenser conveniently allows subsequent dispensing in a plurality of wells.

Alternatively, said fluid is loaded from below, through said access channel 13, for example with a pumping system.

After loading, said fluid is free to flow and occupies the hydrophilic areas present in the integrated microfluidic circuit 25, i.e. the microchannels 5 and the hydrophilic surfaces with relief items 12. Said fluid, thanks to said hydrophilic surfaces with relief items 12, expands above said wells, forming caps of SOL 27 protruding from the micro-droplet plate. When said integrated microfluidic circuit 25 is conveniently filled with said fluid, said fluid is gelled, thus obtaining a micro-droplet plate where the hydrophilic areas of the integrated microfluidic circuit 25 are homogeneously occupied by a gel. In particular, the fluid which is found in correspondence with each of the hydrophilic surfaces with relief items 12 which has formed said caps of SOL 27 gels forming a drop of gel 17.

In a preferred embodiment, said micro-droplet plate is turned face down (FIG. 4A, bottom panel), for the gelling process, where it has been observed that face down gelling advantageously leads to obtaining drops of gel of greater volume than the drops of gel obtained by gelling keeping the micro-droplet plate face up.

In one embodiment, the base of said hydrophobic well 4, in addition to any gaskets, further guarantees the seal, where the fluid remains on said hydrophilic surface with relief items 12, which will be delimited by said hydrophobic rim 21, since the base of said well 4, being hydrophobic, does not attract said fluid which is an aqueous fluid.

Said interconnected cavities 26 favour the complete coverage of said hydrophilic surface with relief items 12. The authors of the present invention have in fact surprisingly demonstrated that the geometry, distribution and number of said relief items 22 on said hydrophilic surface comprised on the upper base 7 of said moving element 3 allow the flow front to advance homogeneously along each direction of said microfluidic circuit 16, until it is completely occupied in its hydrophilic areas. Hydrophilic surfaces without relief items 22, or with said relief items covering too low percentages, less than 10%, or too high, more than 70% of said hydrophilic surfaces, do not allow said homogeneous filling. In said conditions, it has in fact been observed that some of said hydrophilic surfaces are not reached by the fluid.

### Phase 2, Micro-Droplet Insulation (FIG. 4B)

The micro-droplet plate with the hydrophilic areas of the integrated microfluidic circuit 25 homogeneously occupied by fluid in the gel phase is face down and it is conveniently positioned on a cell culture plate 20 containing culture medium 19. The moving elements 3, loaded with said drops of gel 17, even independently of one another, reach the operating position 10, or emerge from said fixed element 2 towards the upper face 14 of the same.

Advantageously, the micro-droplet plate according to the present invention allows the entire micro-droplet plate to be loaded with a single operation, since the formation of said drops is made possible by said hydrophobic rims 21 which delimit the hydrophilic areas. After the transition to gel, the drops of gel 17, thanks to the possibility of said moving elements 3 to move along their own axis, emerge from the plate making the drops of gels accessible for the subsequent seeding step.

Advantageously, said cell culture plate 20 is a multi-well plate, where at least one well of said cell culture plate is conveniently found beneath a moving element 3 loaded with said drop of gel 17.

### Phase 3, Seeding (FIG. 4C)

The drop of gel 17 on said moving element 3 is immersed in the culture medium 19. In one embodiment, the moving element 3, retracting from said operating position 10 to said rest position 9, releases said drop of gel 17 into said culture medium 19. Optionally, said moving element 3 is made to rotate around its own vertical axis 28, thus favouring the release of said drop of gel 17 into said culture medium 19.

Said drop 17, which contains cellular material, for example organoids, is then kept in said culture medium for the entire duration of the treatment. In one embodiment, said micro-droplet plate and said moving elements 3 are kept in the seeding position for the entire duration of the treatment. In this embodiment, the drops of gel 17 remain immersed in the culture medium and adhered to said hydrophilic surfaces with relief items 12.

Advantageously, the moving elements 3 of the micro-droplet plate according to the present invention allow the rapid seeding of homogeneous gel drops in plates.

### Phase 4, Recovery (Optional)

In the event said moving elements 3 have been removed from the seeding position, at the end of the treatment they are brought back into contact with the surface of the culture medium 19. Said drops of gel 17, immersed in said culture medium, will again adhere to said hydrophilic surface with relief items 12.

Where said moving elements 3 had been kept in the seeding position for the entire duration of the treatment, said drops of gel 17 will already be conveniently adhered to said surface with relief items 12.

Optionally, said moving elements 3 are removed, said drops of gel 17 are thus taken from said culture medium 19. Said taken drops of gel 17 are ready to be reused, for example for subsequent seeding in a different culture medium, to be exposed to a different treatment protocol.

Conveniently, the micro-droplet plate according to the present invention comprises M x N wells, where M is equal to or greater than 2 and N is equal to or greater than 2. Preferably, M is equal to or less than 12 and N is equal to or less than 32. In one embodiment, said multi-droplet plate comprises 3 * 3 wells, or 5 * 5, or 7 * 7, or 4 * 8, or 8 * 8. Conveniently, said wells are distributed in said micro-droplet plate maintaining the same distribution geometry observed on 96-well, 384-well, or 1536-well multi-well plates.

Optionally, one or more micro-droplet plates according to the present invention are inserted into a support which houses the number of micro-droplet plates required to seed an entire 96-well, or 384-well, or 1536 multi-well plate.

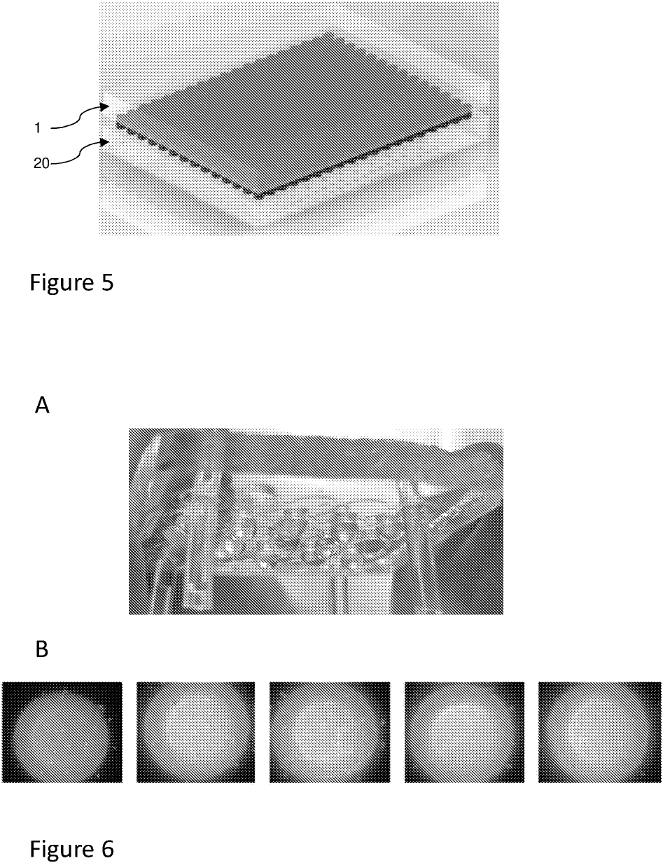

By way of example, FIG. 5 shows a perspective view of a multiwell plate 20, specifically a 384-well plate, on which a series of micro-droplet plates 1 according to the present invention are positioned in a number sufficient to cover the totality of the wells.

In one embodiment, the moving elements 3 are moved in an automated manner, with a controlled and independent movement for each of said moving elements 3.

In one embodiment, said micro-droplet plate comprises blocking structures, suitable for positioning said moving elements 3, even independently of one another, in desired positions, for example in the rest position 9, or in the operating position 10 or, alternatively, in intermediate positions. Conveniently, said multiwell plates have an optically transparent bottom, so that the internal volume of each well can be observed under the microscope.

The following examples are intended to better describe the here proposed solution and are not intended to be limiting in any way. The scope of the invention is defined by the following claims.

### Example 1: Organoid Seeding in 384-Well Plates

The organoids were derived from tumor cells isolated from hepatic metastases of colon carcinoma collected during the surgical procedures provided for patients affected by this disease. Cancer cells dissociated from the original tissue were resuspended in Corning® Matrigel®, a basal membrane preparation in solution extracted from mouse sarcoma, rich in extracellular matrix proteins. The suspension was then plated in the form of drops and after the polymerization it was grown in a culture medium whose composition, consisting of a defined combination of additives and growth factors, was studied and optimized both for the efficient generation of organoids three-dimensional derived from human gastrointestinal carcinoma cells, both for their long-term expansion. Prior to drug treatment, the organoids were collected and dissociated into single cells, then resuspended in Matrigel® at a concentration of 200 cells / µl. The preparation of Matrigel and cells was then plated on traditional 384 multiwells or on micro-droplet″plates, incubated for 20 minutes in a cell culture incubator at 37° C. and 5% CO₂ in order to favor the polymerization of Matrigel® and then immersed in 24 µl of growth medium for each well containing an adequate quantity of drug.

FIG. 6A shows an exemplary photograph of Matrigel® drops containing organoids formed in the micro-droplet plate. FIG. 6B shows exemplary inverted microscope photographs of microwells in which organoids were seeded with the micro-droplet plate.

After seeding, the organoids were exposed to the following treatments, in triplicate:
DMSO: solvent only
BTZ: 1 µM Bortezomib;
OLA: 1 µM Olaparib.

After 96 hours of treatment the vitality of the organoids was measured with the CellTiter-Glo® 3D reagent (Promega) and reported as a percentage referred to the control treated with the solvent alone.

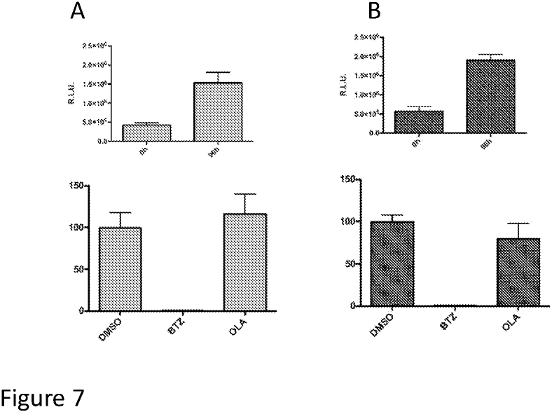

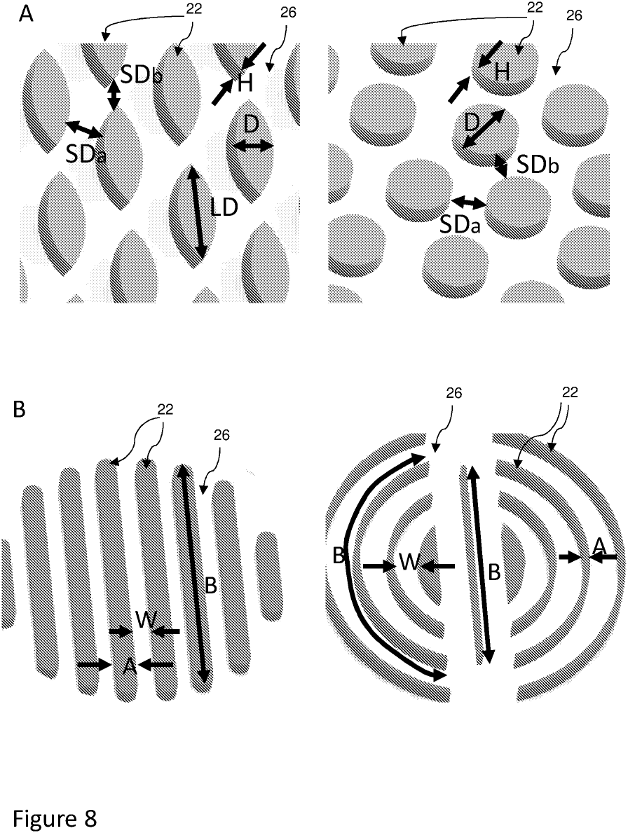

As shown by the graphs in FIG. 7, the cell viability of the organoids (upper panels) at the time of plating and after 96 hours is equivalent in the wells seeded with the traditional method (A) and in those seeded with the multi-droplet plate (B). The same considerations apply to the measurement of the effect of the tested additives. The example shows that the solution according to the present invention is able to give rise to organoid cultures equivalent to those obtained with traditional methods, not on a large scale.

### Numbering

1 micro-droplet plate
2 fixed element
3 moving element
4 hydrophilic wells
5 microchannels
6 hole
7 upper base
8 lower base
9 rest position
10 operating position
11 base support
12 hydrophilic surface with relief items
13 access channel
14 upper face
15 lower face
16 microfluidic circuit
17 drop of gel
18 organoid
19 culture medium
20 cell culture plate
21 hydrophobic rim
22 relief items
23 access well
24 guide channel
25 integrated microfluidic circuit
26 interconnected cavities
27 cap of SOL
28 vertical axis
29 dispenser
30 reservoir

1-13. (canceled)

14. A micro-droplet plate for formation, isolation, seeding, and controlled recovery of volumes between 1 and 400 microliters of fluids, where said fluids are SOL, the micro-droplet plate comprising:
at least one fixed element which is a rigid flat support which has an upper face and a lower face, having, on said upper face, wells connected together by microchannels, said wells being delimited by a hydrophobic rim and crossed by a hole, where said wells and said microchannels define at least one microfluidic circuit; and
moving elements, received in said wells through said holes, which are cylinders having an upper base and a lower base, wherein
said moving elements move, even independently of each other, along their vertical axis, and
the upper base of said moving elements includes a hydrophilic surface with relief items.

15. The micro-droplet plate according to claim 14, wherein said fixed element also comprises at least one access channel which connects the lower face of said fixed element to said at least one microfluidic circuit.

16. The micro-droplet plate according to claim 14, where said moving elements are, even independently of each other, in a rest position or in an operating position, where said rest and operating positions are reached by said moving elements with a movement along their vertical axis, where in said rest position said moving elements have the upper base in a substantially co-planar position with the base of said well, in said operating position said moving elements emerge from said fixed element towards the upper face of the same fixed element.

17. The micro-droplet plate according to one of claim 14, where said moving elements are in a rest position and form an integrated microfluidic circuit which includes said microfluidic circuit and said hydrophilic surfaces with relief items.

18. The micro-droplet plate according to claim 14, wherein said hydrophilic surface with relief items includes relief items distributed in a controlled manner over the entire hydrophilic surface, so as to occupy between 10 and 70% of the overall hydrophilic surface.

19. The micro-droplet plate according to claim 14, wherein the micro-droplet plate is loaded with a SOL which is a formulation which polymerizes producing a biologically active matrix.

20. The micro-droplet plate according to claim 19, wherein said SOL is selected from the group which comprises: formulations based on alginate, chitosan, hyaluronic acid, fibrin, laminin, collagen, Matrigel® (Corning®), or their combinations.

21. A use of the micro-droplet plate according to claim 14 for the formation, isolation, seeding, and/or maintenance in culture, and/or controlled recovery of gel drops.

22. The use according to claim 21, wherein said gel drops are in Matrigel®, said gel drops contain biological material, and said biological material consists of organoids.

23. A method for the formation, the isolation, the seeding and/or maintenance in culture, and/or controlled recovery of gel drops, comprising:
provide the micro-droplet plate according to claim 17;
make available a fluid that is a SOL;
place the at least one of said moving elements in the rest position;
load said micro-droplet plate with said fluid in a liquid phase which by capillarity occupies said integrated microfluidic circuit; and
polymerizing said fluid, obtaining, at each of said hydrophilic surfaces with relief items, drops of gel.

24. The method according to claim 23, wherein said drops of gel contain biological material and said micro-droplet plate is positioned above a cell culture plate which contains culture medium and said drops of gel are seeded in said cell culture plate.

25. The method according to claim 24, wherein at least one of said moving elements is kept in the seeding position for the entire duration of the treatment and said drops of gel remain into the culture medium and adhered to said hydrophilic surfaces with relief items for the entire duration of the culture/treatment.

26. The method according to claim 24, wherein said biological material consists of organoids.
